SEQUENCE LISTING (7 sequences)

SEQ ID NO: 1 - DNA, 1803 bp, Hepatitis C virus
  CDS (1)..(1803)
atggctagcatgtcaatgtcctatacatggacaggcgccctgatcacaccgtgcgctgcggaggaaagcaagctgcccatcaacgcgctgagcaactccttgctgcgtcaccataacctggtctattccacaacatcccgcagtgcaagcctgcggcagaagaaggtcacctttgacagactgcaagtcctggacgatcattaccgggacgtgctcaaggagatgaaggcgaaggcgtccacagtgaaggctaaactgctatctgtagaagaagcatgcaagctgacgcccccgcattcggccaaatccaaatttggctatggggcaaaggacgtccggagcctatccagcagggccgttaaccacatccgctccgtgtggaaggacttgctggaggacactgacacaccaattcagaccaccatcatggcaaaaaatgaggttttctgcgtccaaccagagaaaggaggccgcaaaccagctcgcctcatcgtattcccagacctgggagttcgtgtatgcgagaagatggccctttacgacgtggtttccactcttcctcaggccgtgatgggctcctcatacggattccaatactctcctaagcagcgggtcgagttcctggtgaatacctggaaagcaaagaaatgccctatgggcttctcatatgacacccgctgttttgactcaacggtcactgagaatgacatccgtgttgaggagtcaatttaccaatgttgtgacttggcccccgaagctagacaggccataaggtcgctcacagagcggctctatgtcgggggtcccatgactaactccaaagggcagaactgcggctatcgccggtgccgcgcgagcggcgtgctgacgactagctgcggtaataccctcacatgctacttgaaggccgctgcagcctgtcgagctgccaagctccaggactgcacgatgctcgtgaatggagacgaccttgtcgttatctgtgaaagcgcgggaacccaagaggacgcggcaagcctacgagtcttcacggaggctatgactaggtactctgccccccctggggacccgccccaaccggaatacgacttggagctgataacatcgtgttcctccaatgtgtcggtcgcacacgatgcatctggcaaaagggtgtactacctcacccgtgaccccaccgtcccccttgcgcgggctgcgtgggagacagctaggcacactccagtcaactcctggctaggcaacatcatcatgtatgcgcccactttgtgggcaaggatgattctgatgactcacttcttctccatccttctagcccaggagcaacttgaaaaagccctggattgtcagatctacggggcttgttactccattgagccacttgacctacctcagatcattgaacgactccatggtcttagcgcattttcactccatagttactctccaggtgagatcaatagggtggcttcatgcctcaggaagcttggggtaccacccttgcgagtctggagacatcgggccagaagtgtccgcgctaagttactgtcccagggggggagggccgccatttgtggcaagtacctcttcaactgggcagtaaggaccaagcttaaactcactccaattccggctgcgtcccggctggacttgtccggctggttcgttgctggctacagcgggggagacatatatcacagcctgtctcgtgcccgaccccgctggttcatgttgtgcctactcctactctccgtaggggtaggcatctatctactccccaaccggcatcaccatcaccatcac

SEQ ID NO: 2 - protein, 601 aa, Hepatitis C virus
MASMSMSYTWTGALITPCAAEESKLPINALSNSLLRHHNLVYSTTSRSASLRQKKVTFDRLQVLDDHYRDVLKEMKAKASTVKAKLLSVEEACKLTPPHSAKSKFGYGAKDVRSLSSRAVNHIRSVWKDLLEDTDTPIQTTIMAKNEVFCVQPEKGGRKPARLIVFPDLGVRVCEKMALYDVVSTLPQAVMGSSYGFQYSPKQRVEFLVNTWKAKKCPMGFSYDTRCFDSTVTENDIRVEESIYQCCDLAPEARQAIRSLTERLYVGGPMTNSKGQNCGYRRCRASGVLTTSCGNTLTCYLKAAAACRAAKLQDCTMLVNGDDLVVICESAGTQEDAASLRVFTEAMTRYSAPPGDPPQPEYDLELITSCSSNVSVAHDASGKRVYYLTRDPTVPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRAKLLSQGGRAAICGKYLFNWAVRTKLKLTPIPAASRLDLSGWFVAGYSGGDIYHSLSRARPRWFMLCLLLLSVGVGIYLLPNRHHHHHH

SEQ ID NO: 3 - DNA, 1740 bp, Artificial
  C-terminally truncated HCV NS5B polymerase (C delta 21 NS5B)
atggctagcatgtcaatgtcctatacatggacaggcgccctgatcacaccgtgcgctgcggaggaaagcaagctgcccatcaacgcgctgagcaactccttgctgcgtcaccataacctggtctattccacaacatcccgcagtgcaagcctgcggcagaagaaggtcacctttgacagactgcaagtcctggacgatcattaccgggacgtgctcaaggagatgaaggcgaaggcgtccacagtgaaggctaaactgctatctgtagaagaagcatgcaagctgacgcccccgcattcggccaaatccaaatttggctatggggcaaaggacgtccggagcctatccagcagggccgttaaccacatccgctccgtgtggaaggacttgctggaggacactgacacaccaattcagaccaccatcatggcaaaaaatgaggttttctgcgtccaaccagagaaaggaggccgcaaaccagctcgcctcatcgtattcccagacctgggagttcgtgtatgcgagaagatggccctttacgacgtggtttccactcttcctcaggccgtgatgggctcctcatacggattccaatactctcctaagcagcgggtcgagttcctggtgaatacctggaaagcaaagaaatgccctatgggcttctcatatgacacccgctgttttgactcaacggtcactgagaatgacatccgtgttgaggagtcaatttaccaatgttgtgacttggcccccgaagctagacaggccataaggtcgctcacagagcggctctatgtcgggggtcccatgactaactccaaagggcagaactgcggctatcgccggtgccgcgcgagcggcgtgctgacgactagctgcggtaataccctcacatgctacttgaaggccgctgcagcctgtcgagctgccaagctccaggactgcacgatgctcgtgaatggagacgaccttgtcgttatctgtgaaagcgcgggaacccaagaggacgcggcaagcctacgagtcttcacggaggctatgactaggtactctgccccccctggggacccgccccaaccggaatacgacttggagctgataacatcgtgttcctccaatgtgtcggtcgcacacgatgcatctggcaaaagggtgtactacctcacccgtgaccccaccgtcccccttgcgcgggctgcgtgggagacagctaggcacactccagtcaactcctggctaggcaacatcatcatgtatgcgcccactttgtgggcaaggatgattctgatgactcacttcttctccatccttctagcccaggagcaacttgaaaaagccctggattgtcagatctacggggcttgttactccattgagccacttgacctacctcagatcattgaacgactccatggtcttagcgcattttcactccatagttactctccaggtgagatcaatagggtggcttcatgcctcaggaagcttggggtaccacccttgcgagtctggagacatcgggccagaagtgtccgcgctaagttactgtcccagggggggagggccgccatttgtggcaagtacctcttcaactgggcagtaaggaccaagcttaaactcactccaattccggctgcgtcccggctggacttgtccggctggttcgttgctggctacagcgggggagacatatatcacagcctgtctcgtgcccgaccccgccatcaccatcaccatcac

SEQ ID NO: 4 - protein, 580 aa, Artificial
  C-terminally truncated HCV NS5B polymerase (C delta 21 NS5B)
MASMSMSYTWTGALITPCAAEESKLPINALSNSLLRHHNLVYSTTSRSASLRQKKVTFDRLQVLDDHYRDVLKEMKAKASTVKAKLLSVEEACKLTPPHSAKSKFGYGAKDVRSLSSRAVNHIRSVWKDLLEDTDTPIQTTIMAKNEVFCVQPEKGGRKPARLIVFPDLGVRVCEKMALYDVVSTLPQAVMGSSYGFQYSPKQRVEFLVNTWKAKKCPMGFSYDTRCFDSTVTENDIRVEESIYQCCDLAPEARQAIRSLTERLYVGGPMTNSKGQNCGYRRCRASGVLTTSCGNTLTCYLKAAAACRAAKLQDCTMLVNGDDLVVICESAGTQEDAASLRVFTEAMTRYSAPPGDPPQPEYDLELITSCSSNVSVAHDASGKRVYYLTRDPTVPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRAKLLSQGGRAAICGKYLFNWAVRTKLKLTPIPAASRLDLSGWFVAGYSGGDIYHSLSRARPRHHHHHH

SEQ ID NO: 5 - RNA, 31 nt, Artificial
  HCV NS5B polymerase oligonucleotide template
cgauacucccuuuauauaaccaucaaucgcc

SEQ ID NO: 6 - RNA, 32 nt, Artificial
  HCV NS5B polymerase oligonucleotide template
cgauacucccuuuauauaaccaucaaucgccc

SEQ ID NO: 7 - RNA, 46 nt, Artificial
  HCV NS5B polymerase oligonucleotide template
cucauacgauacucacucuauauaacaaucaaucgccccuuucccc

(19) United States
(12) Patent Application Publication
(10) Pub. No.: US 2004/0209283 A1
(43) Pub. Date: Oct. 21, 2004

(54) Continuous-read assay for the detection of de novo HCV RNA polymerase activity

(75) Inventors: Yoshihiko Yagi, Kalamazoo, MI (US); Michael P. Sheets, Somerset, WI (US); Peter A. Wells, La Jolla, CA (US); John A. Shelly, New Hudson, MI (US); Roger A. Poorman, Portage, MI (US); Dennis E. Epps, Jacksonville, FL (US); Aric G. Morgan, Portage, MI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Pfizer Inc., New York, NY

(21) Appl. No.: 10/712,479

(22) Filed: Nov. 13, 2003

Related U.S. Application Data
(60) Provisional application No. 60/425,981, filed on Nov. 13, 2002.

(51) Int. Cl. C12Q 1/70; C12Q 1/68

(52) U.S. Cl. 435/6; 435/5

(57) ABSTRACT

The invention provides a method for detecting RNA polymerase activity in a continuous-read manner. Specifically, the invention provides a method for detecting the de novo polymerase activity of the Hepatitis C virus (HCV) polymerase, NS5B, in a continuous-read manner. The invention also provides a method of screening for modulators of RNA polymerase activity. More specifically, the invention provides a method of screening for modulators of HCV NS5B activity.

This application claims the benefit of priority from U.S. Provisional App. No. 60/425,981, filed Nov. 13, 2002, the disclosure of which is explicitly incorporated by reference herein.

### FIELD OF THE INVENTION

The invention relates to a method for detecting RNA polymerase activity in a continuous-read manner. Specifically, the invention relates to a method for detecting the de novo polymerase activity of the Hepatitis C virus (HCV) RNA polymerase, NS5B, in a continuous-read manner. The invention also relates to a method of screening for modulators of RNA polymerase activity. More specifically, the invention relates to a method of screening for modulators of HCV NS5B activity.

### BACKGROUND OF THE INVENTION

Hepatitis C virus (HCV) is the major cause of non-A and non-B hepatitis. HCV is acquired mainly through contact with infected blood or blood products. The World Health Organization (WHO) reports that about 80% of newly infected individuals become chronically infected. WHO also estimates that 170 million people worldwide suffer from chronic HCV infection (World Health Organization, 2000). Many infections progress to chronic liver disease, known as chronic hepatitis C. Patients having chronic hepatitis C are at a high risk for serious liver disease such as liver cirrhosis and hepatocellular carcinoma. Current treatment protocols involve antiviral drugs, such as interferon, which can be administered alone or in combination with ribavirin. However, treatment with interferon is only effective in about 10% to 20% of patients, and treatment with interferon combined with ribavirin is effective in about 30% to 50% of patients (World Health Organization, 2000).

No effective vaccine has been developed to prevent HCV infection, largely because the mechanism by which HCV establishes viral persistence has not been thoroughly elucidated and the roles of cellular and humoral immune responses in protecting against HCV infection and disease are not well understood. The lack of an effective protective immune response has hampered both the development of a vaccine and any adequate post-exposure prophylaxis measures. Consequently, the need for effective antiviral interventions is paramount to controlling the disease.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by two viral proteases to produce several structural and non-structural (NS) proteins. The mature nonstructural proteins of HCV are designated NS2, NS3, NS4A, NS4B, NS5A, and NS5B. NS5B is an RNA-dependent RNA polymerase that is involved in the replication of HCV. An attractive strategy for treating HCV infection is to inhibit replication of the virus by inactivating NS5B.

### SUMMARY OF THE INVENTION

The invention provides methods for detecting RNA polymerase activity in a continuous-read manner. Specifically, the invention provides methods for detecting the de novo polymerase activity of the Hepatitis C virus (HCV) polymerase, NS5B, in a continuous-read manner. The invention also provides methods of screening for modulators of RNA polymerase activity. More specifically, the invention provides methods of screening for modulators of HCV NS5B activity.

In one method of the invention, RNA polymerase activity is detected in a continuous-read manner by contacting an RNA polymerase with an oligonucleotide template in a reaction mixture comprising an assay buffer, under conditions in which the RNA polymerase is active; adding a fluorescent dye capable of binding double-stranded nucleic acid molecules to the reaction mixture; and measuring the fluorescence of the reaction mixture.

In another method of the invention, RNA polymerase activity is detected in a continuous-read manner by contacting an RNA polymerase with an oligonucleotide template in a reaction mixture comprising an assay buffer and a fluorescent dye capable of binding double-stranded nucleic acid molecules, under conditions in which the RNA polymerase is active; and measuring the fluorescence of the reaction mixture.

In another method of the invention, HCV NS5B activity is detected in a continuous-read manner by contacting HCV NS5B with an oligonucleotide template in a reaction mixture comprising an assay buffer, under conditions in which the HCV NS5B is active; adding an unsymmetrical cyanine fluorescent dye (such as the dye sold under the trademark PicoGreen® by Molecular Probes, Inc. of Eugene, Oreg.) to the reaction mixture; and measuring the fluorescence of the reaction mixture.

In another method of the invention, HCV NS5B activity is detected in a continuous-read manner by contacting HCV NS5B with an oligonucleotide template in a reaction mixture comprising an assay buffer and an unsymmetrical cyanine fluorescent dye (such as the dye sold under the trademark PicoGreen® by Molecular Probes, Inc. of Eugene, Oreg.), under conditions in which the HCV NS5B is active; and measuring the fluorescence of the reaction mixture.

In another method of the invention, compounds that modulate RNA polymerase activity are determined in a continuous-read manner by contacting an RNA polymerase with an oligonucleotide template in a reaction mixture comprising an assay buffer, under conditions in which the RNA polymerase is active; adding a fluorescent dye capable of binding double-stranded nucleic acid molecules to the reaction mixture; adding a test compound to the reaction mixture; measuring the fluorescence of the reaction mixture; and determining whether the test compound modulates RNA polymerase activity.

In another method of invention, compounds that modulate RNA polymerase activity are determined in a continuous-read manner by contacting an RNA polymerase with an oligonucleotide template in a reaction mixture comprising an assay buffer and a fluorescent dye capable of binding double-stranded nucleic acid molecules, under conditions in which the RNA polymerase is active; adding a test compound to the reaction mixture; measuring the fluorescence of the reaction mixture; and determining whether the test compound modulates RNA polymerase activity.

In another method of the invention, compounds that modulate HCV NS5B activity are determined in a continuous-read manner by contacting HCV NS5B with an oligonucleotide template in a reaction mixture comprising an assay buffer, under conditions in which the HCV NS5B is active; adding an unsymmetrical cyanine fluorescent dye (such as the dye sold under the trademark PicoGreen® by Molecular Probes, Inc. of Eugene, Oreg.) to the reaction mixture; adding a test compound to the reaction mixture; measuring the fluorescence of the reaction mixture; and determining whether the test compound modulates HCV NS5B activity.

In another method of invention, compounds that modulate HCV NS5B activity are determined in a continuous-read manner by contacting HCV NS5B with an oligonucleotide template in a reaction mixture comprising an assay buffer and an unsymmetrical cyanine fluorescent dye (such as the dye sold under the trademark PicoGreen® by Molecular Probes, Inc. of Eugene, Oreg.), under conditions in which the HCV NS5B is active; adding a test compound to the reaction mixture; measuring the fluorescence of the reaction mixture; and determining whether the test compound modulates HCV NS5B activity.

Specific preferred embodiments of the invention will become evident from the following more detailed description of certain preferred embodiments and the claims.

### BRIEF DESCRIPTION OF THE FIGURES

FIGS. 1A-1C illustrate the nucleotide sequence (SEQ ID NO: 1) and deduced amino acid sequence (SEQ ID NO: 2) of a full-length HCV NS5B polymerase (designated FL NS5B).

FIGS. 2A-2C illustrate the nucleotide sequence (SEQ ID NO: 3) and deduced amino acid sequence (SEQ ID NO: 4) of a C-terminally truncated HCV NS5B polymerase (designated C delta 21 NS5B).

FIG. 3 is a graph showing a time course for an NS5B-catalyzed reaction containing 56 nM PicoGreen®. Relative fluorescent units (RFU) were plotted with respect to elapsed time and the data fit to a first-order equation for an increasing signal. The results shown for each time point represent the average of three measurements.

FIG. 4 is a histogram depicting the fluorescent enhancement of PicoGreen® upon binding to double-stranded RNA. The results shown in the histogram for single-stranded and double-stranded RNA at each concentration represent the average of two measurements.

FIGS. 5A-5C are histograms of the kinetic parameters: span (FIG. 5A), kₑₓₚ (FIG. 5B), and calculated vₒ (FIG. 5C), showing the effects of increasing PicoGreen® concentrations on NS5B-dependent reaction kinetics. The results shown in each histogram represent the average of two measurements.

FIG. 6 is a graph showing an C delta 21 NS5B enzyme-catalyzed reaction containing 600 nM PicoGreen®. RFU were plotted with respect to elapsed time and the data fit to an integrated first-order equation.

FIG. 7 is a graph showing a first-order full-length NS5B enzyme-catalyzed reaction containing 600 nM PicoGreen® and 75 μg/ml large unilamellar vesicles. RFU were plotted with respect to elapsed time and the data fit to an integrated first-order equation.

FIG. 8 is a graph showing a C delta 21 NS5B enzyme-catalyzed reaction containing 400 nM SYBR® Green I. RFU were plotted with respect to elapsed time and the data fit to an integrated first-order equation. The results shown for each time point represent the average of four measurements.

FIG. 9 is a graph showing a C delta 21 NS5B enzyme-catalyzed reaction containing RiboGreen® (1:580 dilution of stock dye). RFU were plotted with respect to elapsed time and the data fit to an integrated first-order equation. The results shown for each time point represent the average of four measurements.

### DETAILED DESCRIPTION OF THE PREFERRED EMBODIMENTS

Standard techniques were used for recombinant DNA manipulations, oligonucleotide synthesis, tissue culture, and transformation (e.g., electroporation, lipofection). Enzymatic reactions and purification techniques were performed according to manufacturers' specifications, as commonly accomplished in the art, or as described herein. The techniques and procedures were generally performed according to conventional methods well known in the art and as described in various general and more specific references that are cited and discussed throughout the present specification. See e.g., Sambrook et al., Molecular Cloning: A Laboratory Manual (3d ed. 2001), which is incorporated herein by reference. Standard techniques can be used for chemical syntheses, chemical analyses, pharmaceutical preparation, formulation, and delivery, and treatment of patients.

Unless otherwise required by context, singular terms shall include pluralities and plural terms shall include the singular.

As utilized in accordance with the present disclosure, the terms used herein, unless otherwise indicated, have ordinary meanings as understood by those skilled in the art.

The invention provides methods of detecting de novo RNA polymerase activity and methods of screening for modulators of RNA polymerase activity in a continuous-read manner. Any RNA polymerase can be used in the method of the invention, including, but not limited to, RNA polymerase I, II, III, and viral RNA polymerases. The RNA polymerase used in the methods of the invention can be either recombinant or endogenous.

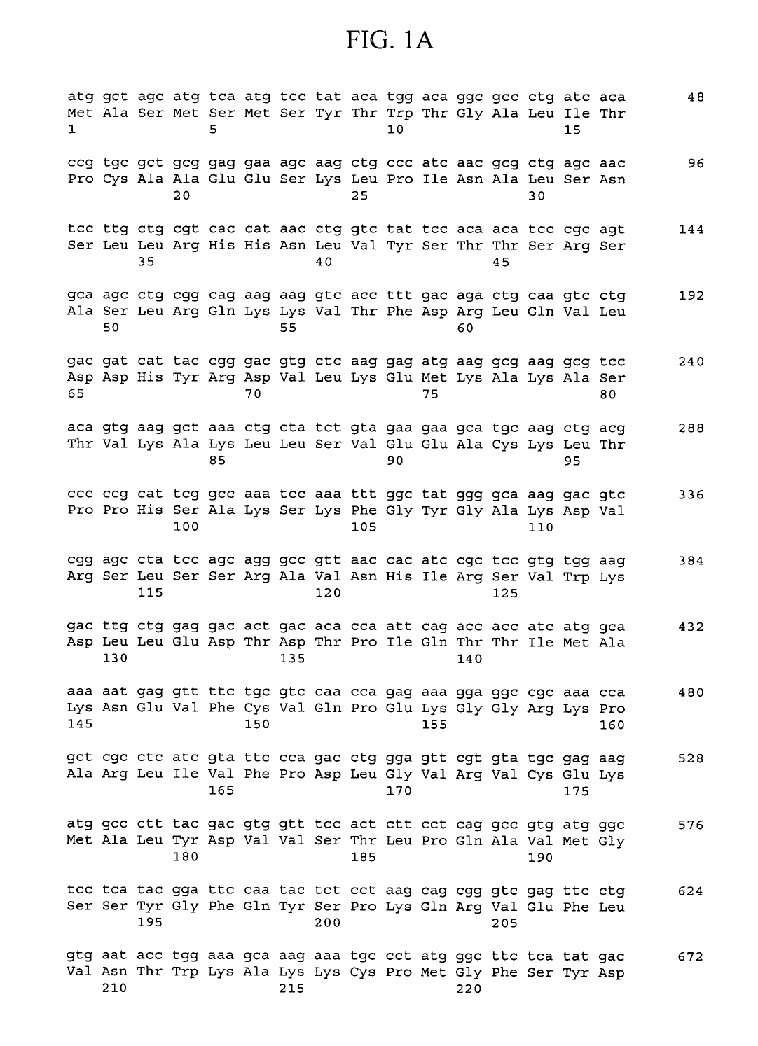

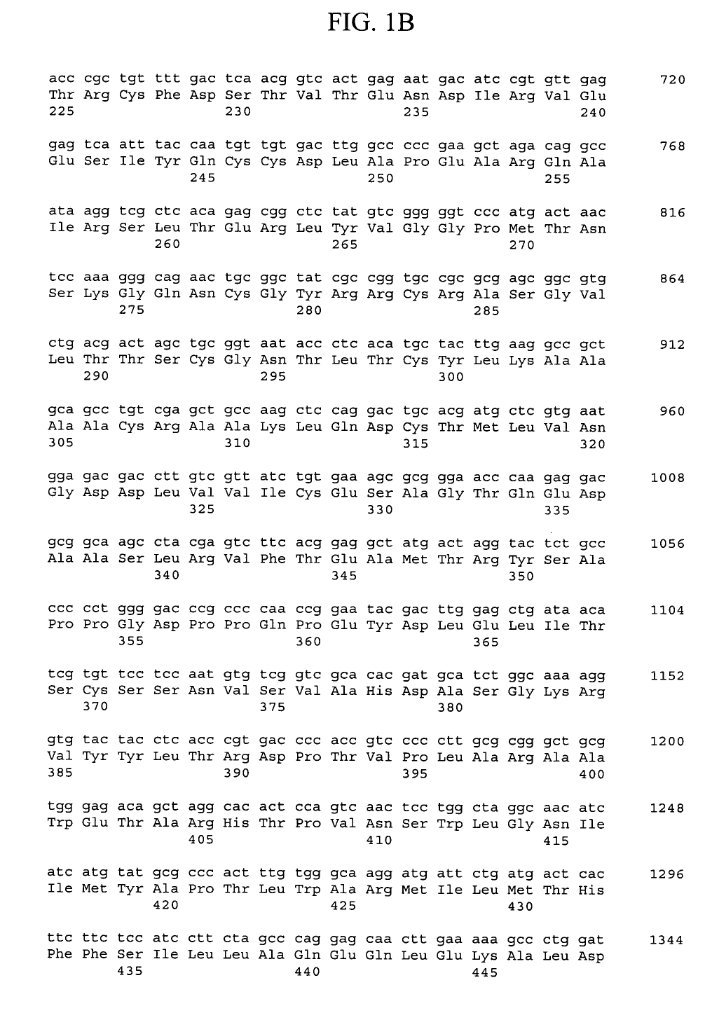

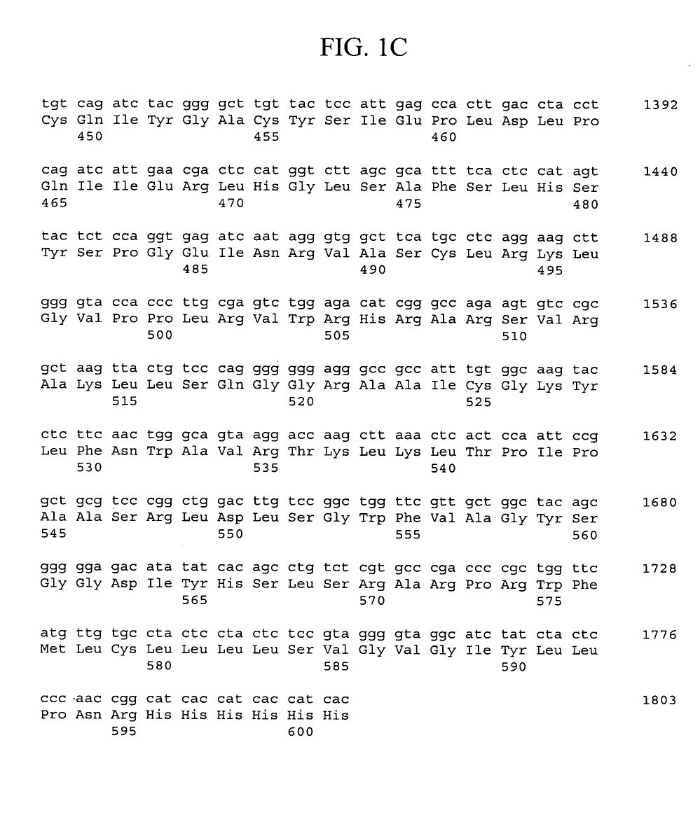

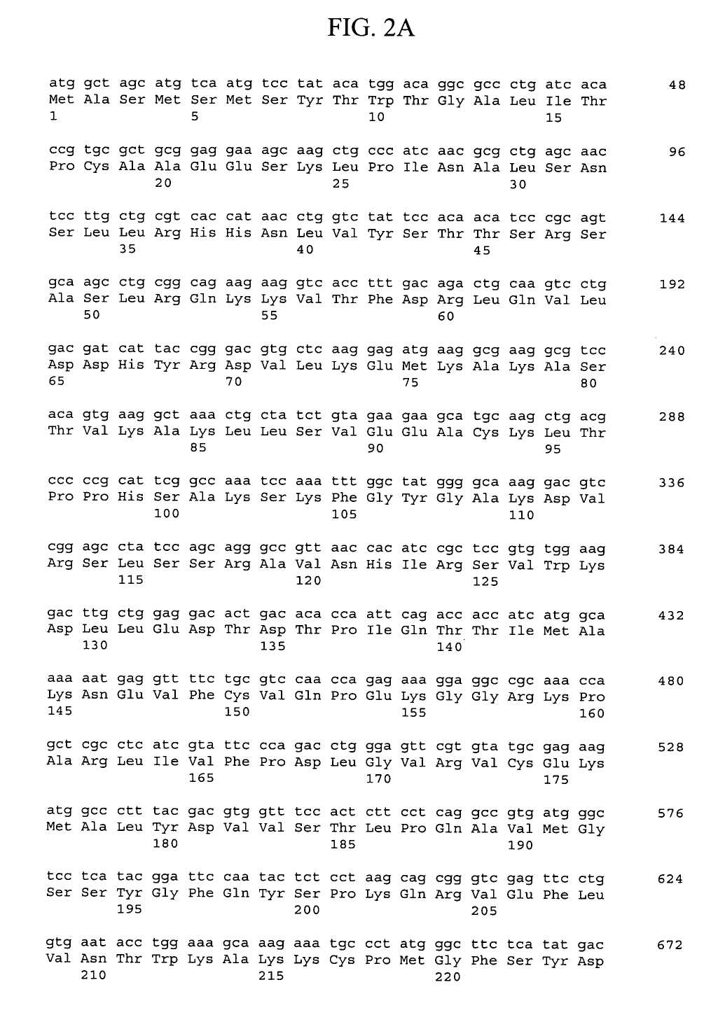

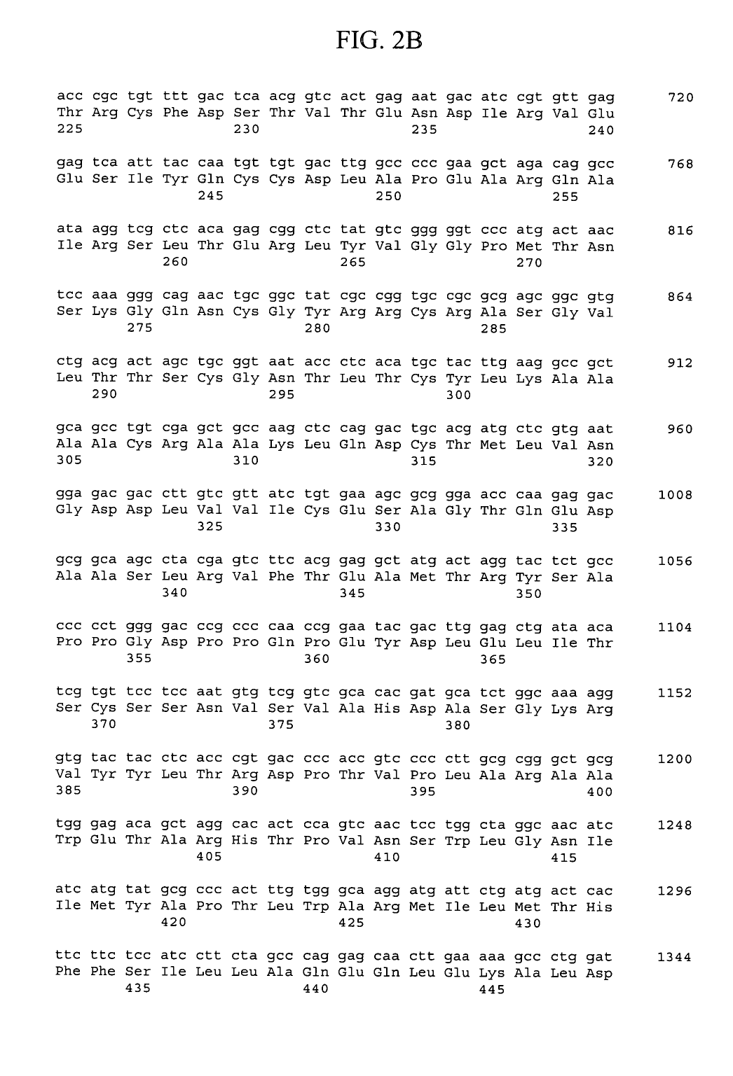

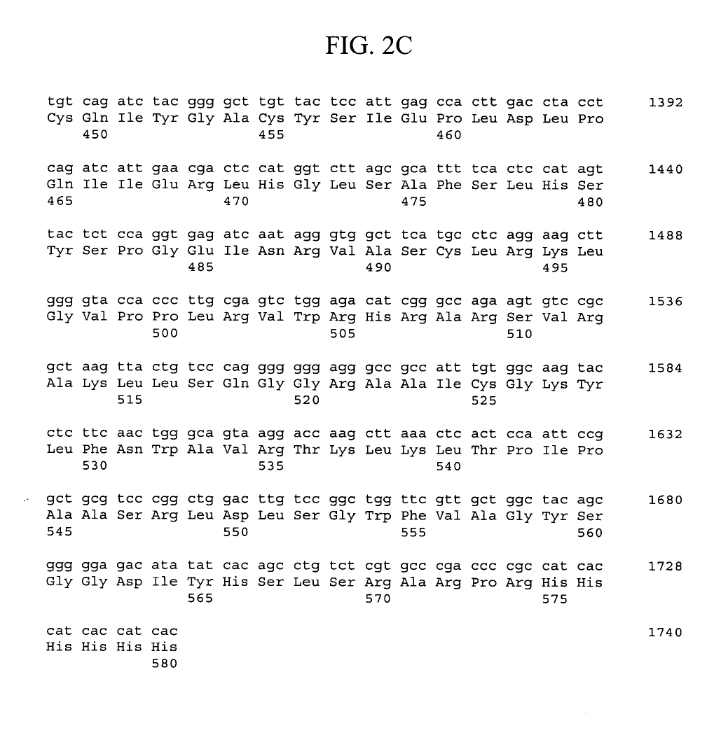

In preferred embodiments of the methods of the invention, the RNA polymerase is the Hepatitis C virus (HCV) polymerase, NS5B. In one preferred embodiment, the RNA polymerase is a recombinant HCV NS5B, such as the recombinant HCV NS5B polymerase shown in FIGS. 1A-1C and designated as FL NS5B. In another preferred embodiment, the RNA polymerase is a truncated HCV NS5B polymerase, such as the C-terminally truncated HCV NS5B polymerase shown in FIGS. 2A-2C and designated as C delta 21 NS5B. Other NS5B variants that retain polymerase activity can be used in the methods of the invention. When FL NS5B polymerase is used in the methods of the invention, the reaction mixture should be supplemented with large unilamellar vesicles (MacDonald et al., 1991, Biochimica et Biophysica Acta, 1061:297-303) or cellular microsomes in order to obtain a level of polymerase activity equivalent to that obtained with C delta 21 NS5B.

A “continuous-read” assay as described herein refers to a method of detecting RNA synthesis without the need to “stop” the reaction. A “stop” or “stopped” reaction, also referred to herein as an “end-point” assay, is one in which RNA synthesis has been terminated. Traditional methods of detecting RNA synthesis involve end-point assays, in which synthesis is detected only at specific time points. A continuous-read kinetic assay, as described herein, yields more information relating to the mechanism of modulating or inhibiting RNA polymerase activity compared with a stopped reaction. For example, the continuous-read assay of the invention provides the ability to rapidly identify reversible and nonreversible inhibitors of RNA polymerase activity.

In certain embodiments, the methods of the invention comprise contacting an RNA polymerase with an oligonucleotide template in a reaction mixture comprising an assay buffer. The term “contacting” as used herein refers to any action that permits an RNA polymerase to come in contact with an oligonucleotide template, for example, by mixing the polymerase and oligonucleotide together in a well of an assay plate. An oligonucleotide template preferably comprises ribonucleotides, and has a sequence that permits replication by an RNA polymerase. Suitable oligonucleotide templates for use with particular RNA polymerases are well known in the art. For example, it is known in the art that an oligonucleotide template for use in assaying de novo HCV NS5B polymerase must contain two or more C residues or two or more U residues at the 3′ terminus. Suitable oligonucleotide templates for use in assaying de novo HCV NS5B polymerase activity include the following:

| | |
|---|---|
| 5′-C-G-A-U-A-C-U-C-C-C-U-U-U-A-U-A-U-A-A-C-C-A-U-C-A-A-U-C-G-C-C-3′; | (SEQ ID NO: 5) |
| 5′-C-G-A-U-A-C-U-C-C-C-U-U-U-A-U-A-U-A-A-C-C-A-U-C-A-A-U-C-G-C-C-C 3′; | (SEQ ID NO: 6) |
| and | |
| 5′-C-U-C-A-U-A-C-G-A-U-A-C-U-C-A-C-U-C-U-A-U-A-U-A-A-C-A-A-U-C-A-A-U- | (SEQ ID NO: 7) |
| C-G-C-C-C-C-U-U-U-C-C-C-C-3′. | |

Generally, the methods of the invention are conducted under conditions as described in the Examples below. However, any conditions in which the RNA polymerase is active can be used.

In the methods of the invention, a fluorescent dye capable of binding double-stranded nucleic acid molecules is used. Preferably, the fluorescent dye is an unsymmetrical cyanine fluorescent dye. A suitable unsymmetrical cyanine fluorescent dye is the dye obtained from Molecular Probes, Inc. (Eugene, Oreg.) in February, 2001, having catalog number P-7581, and being sold under the trademark PicoGreen®. Under preferred assay conditions, this dye is excited at between 475 nm and 495 m and dye fluorescence is detected at between 518 nm and 542 nm. Another suitable unsymmetrical cyanine fluorescent dye is the dye obtained from Molecular Probes, Inc. in January, 2002 having catalog number R-11491, and being sold under the trademark RiboGreen®. Still another suitable fluorescent dye is the dye obtained from Molecular Probes, Inc. in October, 1997, having catalog number S-7563, and being sold under the trademark SYBR® Green I. Preferably, the fluorescent dye used in the continuous-read assay of the invention is the dye being sold under the trademark PicoGreen®.

Seville et al. describe the use of PicoGreen® for detecting E. coli DNA polymerase III holoenzyme activity in a continuous-read manner (Seville et al., 1996, BioTecniques 21:664-72). However, those investigators did not detect enzyme activity in similar assays using HIV reverse transcriptase. In view of the observations made by Seville et al. when using HIV reverse transcriptase (a viral polymerase) in continuous-read assays, the results of the continuous-read assays described herein are unexpected.

In certain embodiments, the methods of the invention can be used to identify modulators or inhibitors of RNA polymerase activity in a continuous-read manner. In preferred embodiments, the methods of the invention are used to identify modulators or inhibitors of the HCV NS5B polymerase.

The term “inhibitor” is used herein to refer to a compound that can block or interfere with RNA polymerase activity. A “modulator of RNA polymerase activity” can be, for example, an agonist or an antagonist of RNA synthesis. An “agonist” of RNA polymerase activity is a compound that initiates or increases the activity of the RNA polymerase. An “antagonist” of RNA polymerase activity is a compound that reduces the activity of the RNA polymerase.

In other embodiments of the invention, the RNA polymerase modulators or inhibitors identified using the methods of the invention are used to treat patients with a particular disease or condition. In preferred embodiments, the HCV NS5B modulators or inhibitors identified using the methods of the invention are used to treat patients with a disease or condition associated with HCV. HCV-associated diseases and conditions include, but are not limited to, antiphospholipid antibody syndrome, autoimmune hepatitis, thrombocytopenia, bone mineral diseases (such as osteosclerosis, osteoporosis, and hepatic osteodystrophy), carcinomas (such as head-neck squamous cell carcinoma and hemangioma), cardiovascular diseases, diabetes, ocular disorders (such as optic neuropathy), fibromyalgia, renal dysfunction, lymphomas, lymphoproliferative disorders, metabolic disorders, arthritis, sleep disorders, and thyroid disorders.

In other embodiments, the methods of the invention can be combined with end-point assays to confirm the ability of pre-screened compounds to modulate RNA polymerase activity. For example, high-throughput screening (HTS) using end-point biochemical or cell-based assays can be used to screen large libraries of chemical compounds or natural products for inhibitors of NS5B polymerase activity. See Sundberg, 2000, Current Opinion in Biotechnology 11:47-53 (reviewing HTS methods); Hertzberg et al., 2000, Current Opinion in Chemical Biology 4:445-51 (reviewing HTS methods). Chemical or natural products that show activity in HTS using end-point (stopped) assays can then be evaluated using the PicoGreen® continuous-read assay and information obtained regarding the mechanism of inhibition.

In preferred embodiments, the invention provides pharmaceutical compositions comprising a therapeutically effective amount of one or a plurality of the NS5B polymerase inhibitors or modulators of the invention together with a pharmaceutically acceptable diluent, carrier, solubilizer, emulsifier, preservative, or adjuvant. Preferably, acceptable formulation materials are nontoxic to recipients at the dosages and concentrations employed. In preferred embodiments, pharmaceutical compositions comprising a therapeutically effective amount of NS5B polymerase inhibitors or modulators are provided. Pharmaceutical compositions can be prepared as described, for example, in Remington's Pharmaceutical Sciences (A. R. Gennaro, ed., 18th ed. 1990).

The term “pharmaceutical composition” as used herein refers to a chemical compound or composition capable of inducing a desired therapeutic effect when properly administered to a patient. The term “patient” includes human and animal subjects. The term “therapeutically effective amount” refers to the amount of a compound identified in a screening method of the invention determined to produce a therapeutic response in a mammal. Such therapeutically effective amounts are readily ascertained by one of ordinary skill in the art.

The following examples, including the experiments conducted and results achieved, are provided for illustrative purposes only and are not to be construed as limiting the present invention.

### EXAMPLE 1

### Measuring NS5B-Dependent De Novo Conversion Of Single-Stranded Template RNA to Double-Stranded Product

To detect RNA polymerase activity using the methods of the invention, reactions containing a fluorescent dye, a recombinant NS5B polymerase, and an oligonucleotide template were prepared. An unsymmetrical cyanine dye sold under the trademark PicoGreen® was utilized as the fluorescent dye in this assay. This dye, which is manufactured and marketed by Molecular Probes, Inc. as a reagent for double-stranded DNA quantitation, exhibits an approximately 2000-fold fluorescent enhancement upon binding to double-stranded DNA, and a nearly 1100-fold fluorescent enhancement when bound to ribosomal RNA, over dye alone (Singer et al., 1997, Analytical Biochem. 249:228-38).

A C-terminally truncated NS5B polypeptide, designated as C delta 21 NS5B, was utilized as the recombinant NS5B polypeptide in this assay. The nucleic acid sequence encoding C delta 21 NS5B was engineered to add four amino acid residues at the amino-terminal end of the encoded polypeptide and to replace the 21-amino acid residue hydrophobic tail at the carboxyl-terminal end with a polyhistidine tag. FIGS. 2A-2C illustrate the nucleotide sequence encoding C delta 21 NS5B and the deduced amino acid sequence of C delta 21 NS5B.

To generate a suitable template, an oligonucleotide having the nucleotide sequence 5′-C-G-A-U-A-C-U-C-C-C-U-U-U-A-U-A-U-A-A-C-C-A-U-C-A-A-U-C-G-C-C-3′ (SEQ ID NO: 5) was prepared by phosphoramidite solid-phase synthesis technology (Matteucci et al., 1981, J. Am. Chem. Soc. 103:3185-91; Beaucage et al., 1981, Tetrahedron Letters 25:1859-62). While NS5B activity can be readily detected by the methods of the invention using unmodified oligonucleotide templates, the 5′ and 3′ ends of the oligonucleotide synthesized above were modified by conjugation with biotin and a deoxy-C terminator, respectively. The resulting oligonucleotide template was designated NR-2.

Reactions were prepared by first placing 0.5 μL of 100% DMSO into the wells of an assay plate (Corning, black, 384-well, NBS #3654). In reactions in which modulators or inhibitors of RNA polymerase activity are to be identified, a test compound may be added to the DMSO. A solution (Solution-1) containing 162 nM of C delta 21 NS5B and 28 nM NR-2 in an assay buffer comprising 20 mM Tris-HCl, pH 7.5; 100 mM ammonium acetate; 2 mM MnCl₂; 10 mM DTT; and 2 mM CHAPS, was then prepared and incubated at ambient temperature for a minimum of 15 minutes. During this incubation, a second solution (Solution-2) was prepared by mixing 76 nM of PicoGreen® with 2.7 μM UTP, 2.7 μM CTP, 2.7 μM ATP, 67 μM GTP in the assay buffer that is described above. Next, 10 μL of Solution-1 was added to the wells of the assay plate containing DMSO and the plate was incubated for 15 minutes at ambient temperature. Reactions were initiated by adding 30 μL of Solution-2 to the DMSO/Solution-1 mixture. The de novo (unprimed) reactions were run for about 1.5 hours after the addition of Solution-2.

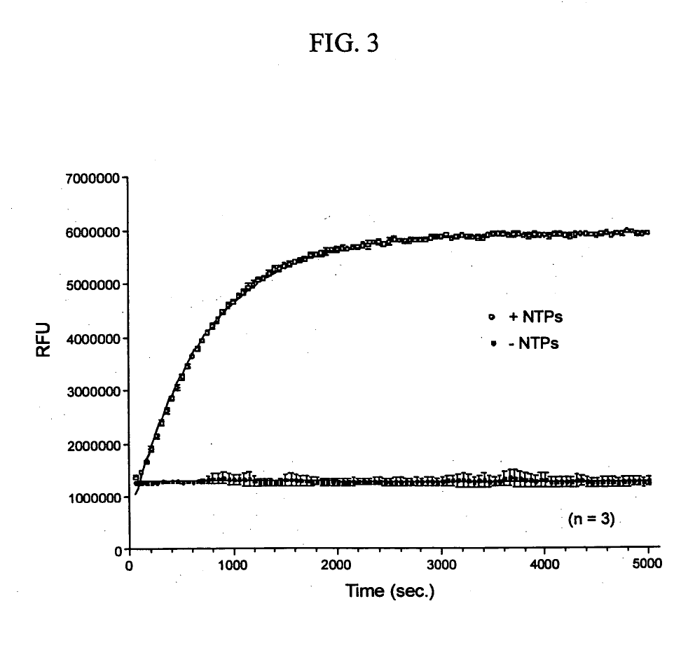

The reactions were monitored on a LJL Analyst (Molecular Devices Corp.; Sunnyvale, Calif.), which excited the PicoGreen® at 485 nm and detected PicoGreen® fluorescence at 530 nm. As shown in FIG. 3, the assay reaction with four nucleotide triphosphates showed a time dependent increase in fluorescence which could be fit to a first order rate equation (R²=0.995). Control reactions (i.e., reactions performed in the absence of nucleotide triphosphates) exhibited no increase in fluorescence.

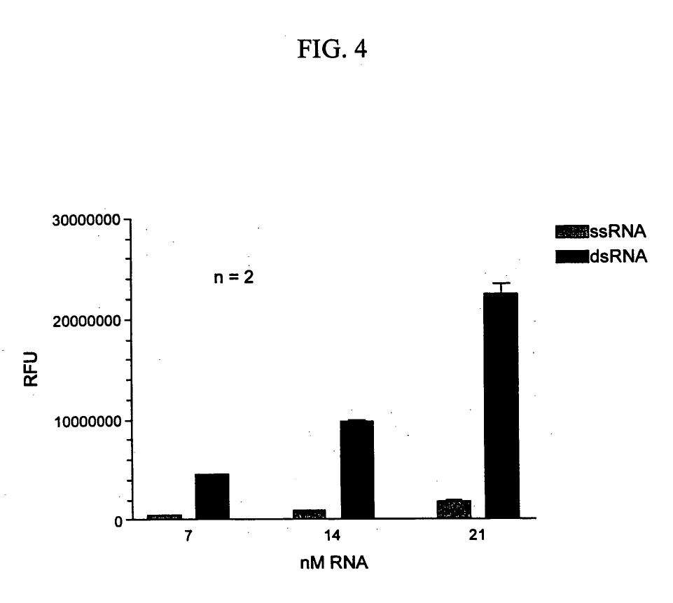

To confirm that the kinetic results were double-stranded RNA dependent, an RNA molecule comprising a nucleotide sequence complimentary to that of the NR-2 oligonucleotide template was prepared and annealed to the NR-2 template using standard conditions for RNA-RNA hybridization. Reactions were then performed using 600 nM PicoGreen® and 7 nM, 14 nM, or 21 nM of single or double-stranded NR-2 (FIG. 4). All other reaction conditions were as described above. The fluorescence intensity (FI) observed with double-stranded NR-2 was 3 to 12 fold higher than the Fl observed with single-stranded NR-2.

### EXAMPLE 2

### Effect of Various Assay Conditions on NS5B Polymerase Activity

To assess the effect of buffer component concentration on the binding of PicoGreen® to double-stranded NR-2, mixtures containing 7 nM double-stranded NR2, 600 nM PicoGreen®, and varying concentrations of the buffer components were prepared, and the fluorescence intensities of these mixtures were measured. These mixtures were prepared using between 4 fold less to 1.25 to 2 fold more of the individual buffer components (relative to the amount of the components used in the reactions described in Example 1). The results are shown in Table 1.

TABLE I

| | Final Concentration | Fluorescence Intensity |
|---|---|---|
| Component | (mM) | (% of control ± SD; n = 2) |
| Buffer Alone (Control) | — | 100 |
| MnCl₂ | 4 | 45 ± 2 |
| | 2 | 65 ± 1 |
| | 1 | 82 ± 1 |
| | 0.5 | 96 ± 2 |
| Ammonium Acetate | 125 | 89 ± 2 |
| | 100 | 95 ± 1 |
| | 50 | 109 ± 1 |
| | 25 | 103 ± 1 |
| | 12.5 | 105 ± 1 |
| CHAPS | 4 | 102 ± 1 |
| | 2 | 96 ± 1 |
| | 1 | 96 ± 1 |
| | 0.5 | 98 ± 3 |
| DTT | 20 | 96 ± 3 |
| | 10 | 100 ± 1 |
| | 5 | 100 ± 2 |
| | 2.5 | 98 ± 5 |

Neither DTT nor chaps affected the binding of PicoGreen® to the double-stranded template, at the concentrations tested. MnCl₂ was found to inhibit the binding of PicoGreen® to the template by approximately 35% and 55% at 2 mM and 4 mM, respectively, and ammonium acetate was found to inhibit PicoGreen® binding by 10% and 5% at 125 mM and 100 mM, respectively. However, 100 mM ammonium acetate appeared to enhance NS5B activity, and this amount of ammonium acetate was used in all subsequent assays.

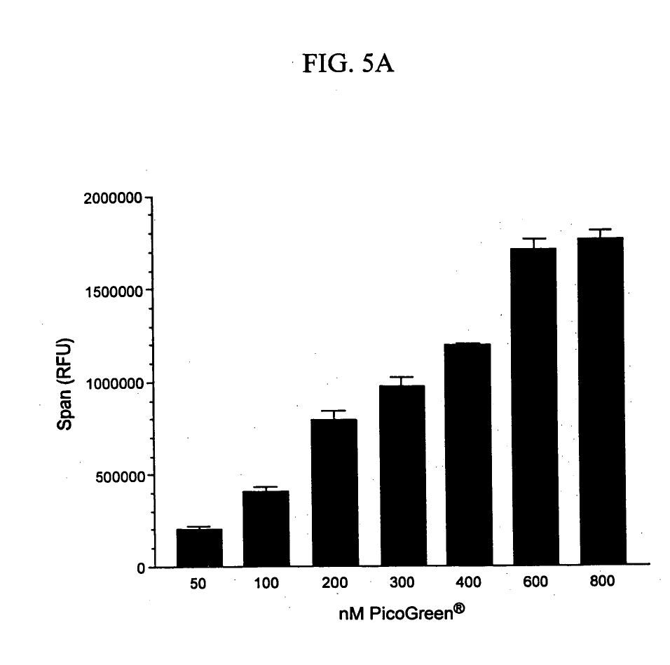

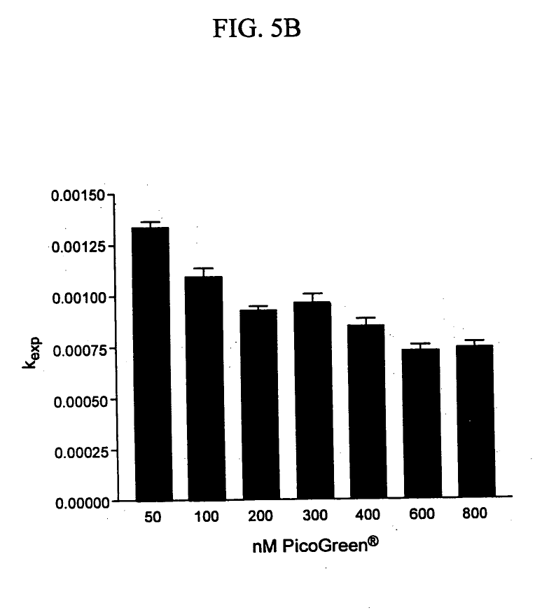

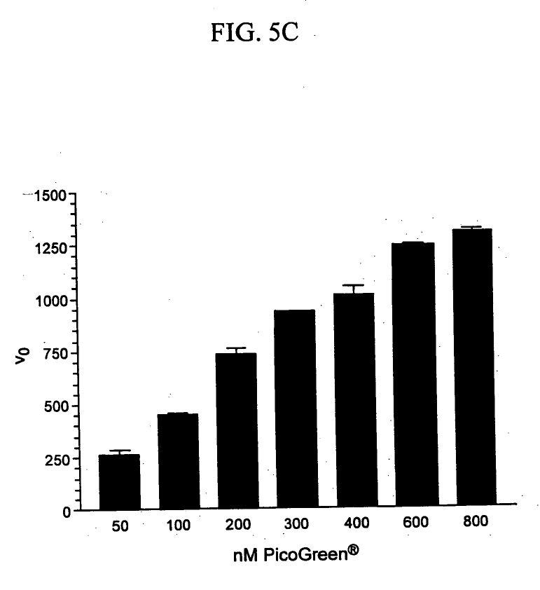

The effect of increasing concentrations of PicoGreen® on reaction kinetics was also assessed. As the concentration of PicoGreen® was increased from 50 nM to 600 nM, the span of the first-order reaction increased, and appeared to plateau when between 600 and 800 nM of PicoGreen® was used (FIG. 5A). The experimental rate constant (kₑₓₚ) of the first-order reaction decreased with increasing PicoGreen® concentrations and appeared to plateau at between 600 and 800 nM of PicoGreen® with slightly less than a two-fold decrease in kₑₓₚ at 600 nM PicoGreen® (FIG. 5B). The calculated initial velocity (v₀=span x kₑₓₚ) increased as the concentration of PicoGreen® was increased from 50 to 600 nM, and appeared to plateau at between 600 and 800 nM PicoGreen® (FIG. 5C).

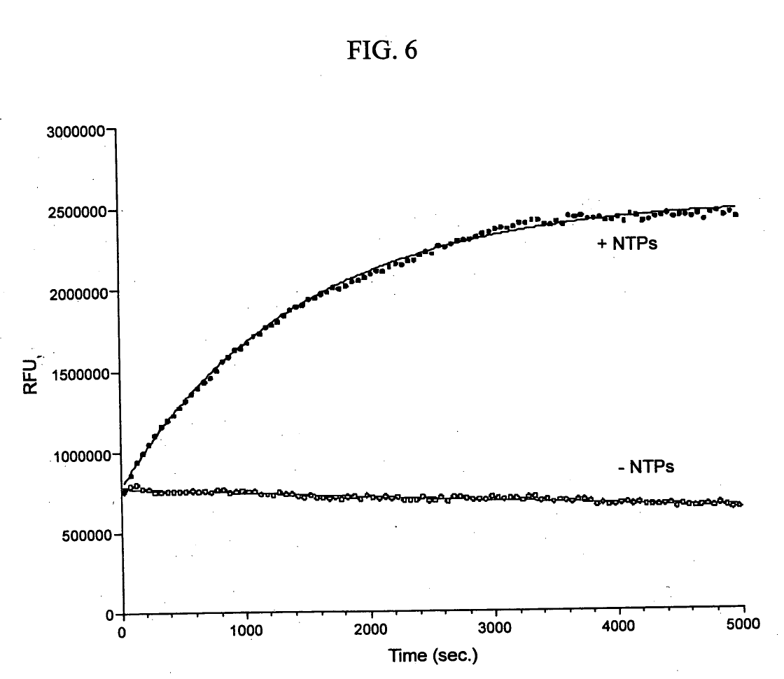

The results of these experiments indicated that 40 nM NS5B, 7 nM NR-2, and 600 nM PicoGreen® provided reasonable and reliable results for measuring NS5B-dependent activity (FIG. 6).

### EXAMPLE 3

### Measuring Full-length NS5B-dependent De Novo Conversion of Single-stranded Template RNA to Double-stranded Product in the Presence of Large Unilamellar Vesicles

Large unilamellar vesicles or other forms of lipid bilayers, such as microsomes, can be used to stabilize enzymes that contain transmembrane domains, as is the case with the HCV full-length NS5B enzyme.

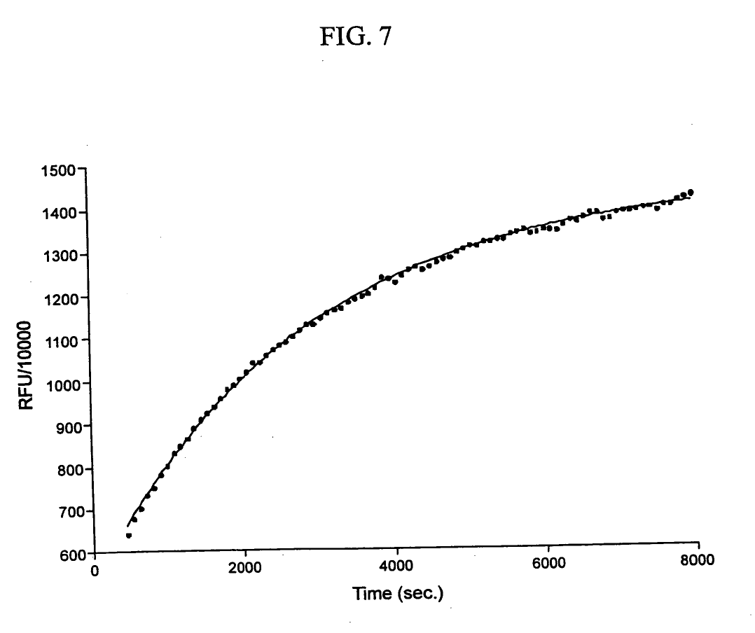

To assess the effect of large unilamellar vesicles on PicoGreen® binding, reactions were performed as described in Example 1, except for the addition of 80 nM full-length NS5B, 20 nM of the oligonucleotide template set forth in SEQ ID NO: 6, and 75 μg/mL of large unilamellar vesicles. Unilamellar vesicles were prepared according to the method of MacDonald et al., 1991, Biochimica et Biophysica Acta, 1061:297-303). When reactions mediated by full-length NS5B were supplemented with large unilamellar vesicles, a time dependent increase in fluorescence was observed (FIG. 7). The presence of stabilizing lipid membranes did not significantly interfere with the fluorescence or nucleic acid binding properties of PicoGreen®.

### EXAMPLE 4

Using Other Fluorescent Dyes to Measure RNA polymerase Activity

To assess the feasibility of using fluorescent dyes other than PicoGreen® in the continuous read fluorescence assay of the invention, reactions were performed using SYBR® Green I (Molecular Probes) or RiboGreen® (Molecular Probes).

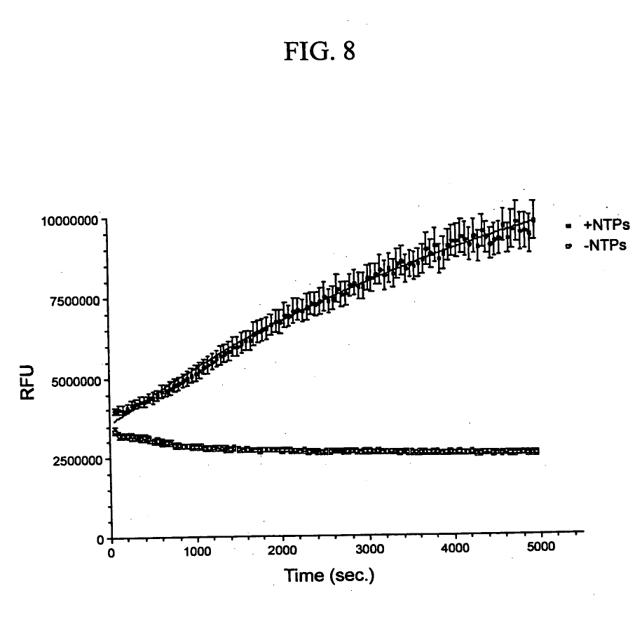

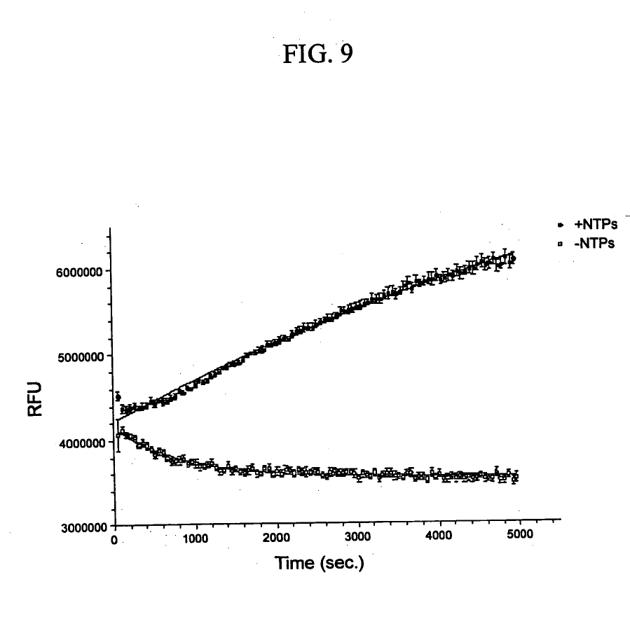

Reactions containing 400 nM SYBR® Green I, 40 nM NS5B C delta 21, and 7 nM NR-2 were found to proceed in a similar manner to those containing PicoGreen®, yielding an increase in the fluorescence signal over time and a usable signal to background ratio (FIG. 8). Similar results were obtained with reactions containing RiboGreen® (1/580 dilution of stock dye) in place of PicoGreen® (FIG. 9).

It should be understood that the foregoing disclosure emphasizes certain specific embodiments of the invention and that all modifications or alternatives equivalent thereto are within the spirit and scope of the invention as set forth in the appended claims.

We claim:

1. A method of detecting RNA polymerase activity in a continuous-read manner, comprising the steps of:
(a) contacting an RNA polymerase with an oligonucleotide template in a reaction mixture comprising an assay buffer, under conditions in which the RNA polymerase is active;
(b) adding a fluorescent dye capable of binding double-stranded nucleic acid molecules to the reaction mixture;
(c) measuring the fluorescence of the reaction mixture.

2. The method of claim 1, wherein the RNA polymerase is a recombinant RNA polymerase.

3. The method of claim 1, wherein the RNA polymerase is the Hepatitis C virus (HCV) polymerase, NS5B.

4. The method of claim 3, wherein the NS5B polymerase is a recombinant NS5B polymerase.

5. The method of claim 4, wherein the recombinant NS5B polymerase comprises an amino acid sequence as set forth in SEQ ID NO: 2.

6. The method of claim 5, wherein the reaction mixture further comprises large unilamellar vesicles.

7. The method of claim 4, wherein the recombinant NS5B polymerase comprises an amino acid sequence as set forth in SEQ ID NO: 4.

8. The method of claim 1, wherein the fluorescent dye is an unsymmetrical cyanine fluorescent dye.

9. The method of claim 8, wherein the unsymmetrical cyanine fluorescent dye is excited at between 475 nm and 495 nm and dye fluorescence is detected at between 518 nm and 542 nm.

10. A method of detecting RNA polymerase activity in a continuous-read manner, comprising the steps of:
(a) contacting an RNA polymerase with an oligonucleotide template in a reaction mixture comprising an assay buffer and a fluorescent dye capable of binding double-stranded nucleic acid molecules, under conditions in which the RNA polymerase is active; and
(b) measuring the fluorescence of the reaction mixture.

11. The method of claim 10, wherein the RNA polymerase is a recombinant RNA polymerase.

12. The method of claim 10, wherein the RNA polymerase is the Hepatitis C virus (HCV) polymerase, NS5B.

13. The method of claim 12, wherein the NS5B polymerase is a recombinant NS5B polymerase.

14. The method of claim 13, wherein the recombinant NS5B polymerase comprises an amino acid sequence as set forth in SEQ ID NO: 2.

15. The method of claim 14, wherein the reaction mixture further comprises large unilamellar vesicles.

16. The method of claim 13, wherein the recombinant NS5B polymerase comprises an amino acid sequence as set forth in SEQ ID NO: 4.

17. The method of claim 10, wherein the fluorescent dye is an unsymmetrical cyanine fluorescent dye.

18. The method of claim 17, wherein the unsymmetrical cyanine fluorescent dye is excited at between 475 nm and 495 nm and dye fluorescence is detected at between 518 nm and 542 nm.

19. A method of screening for compounds that modulate RNA polymerase activity in a continuous-read manner, comprising the steps of:
(a) contacting an RNA polymerase with an oligonucleotide template in a reaction mixture comprising an assay buffer, under conditions in which the RNA polymerase is active;
(b) adding a fluorescent dye capable of binding double-stranded nucleic acid molecules to the reaction mixture;
(c) adding a test compound to the reaction mixture;
(d) measuring the fluorescence of the reaction mixture; and
(e) determining whether the test compound modulates RNA polymerase activity.

20. The method of claim 19, wherein the RNA polymerase is a recombinant RNA polymerase.

21. The method of claim 19, wherein the RNA polymerase is the Hepatitis C virus (HCV) polymerase, NS5B.

22. The method of claim 21, wherein the NS5B polymerase is a recombinant NS5B polymerase.

23. The method of claim 22, wherein the recombinant NS5B polymerase comprises an amino acid sequence as set forth in SEQ ID NO: 2.

24. The method of claim 23, wherein the reaction mixture further comprises large unilamellar vesicles.

25. The method of claim 22, wherein the recombinant NS5B polymerase comprises an amino acid sequence as set forth in SEQ ID NO: 4.

26. The method of claim 19, wherein the fluorescent dye is an unsymmetrical cyanine fluorescent dye.

27. The method of claim 26, wherein the unsymmetrical cyanine fluorescent dye is excited at between 475 nm and 495 nm and dye fluorescence is detected at between 518 nm and 542 nm.

28. The method of claim 19, wherein the compound that modulates the RNA polymerase activity is an antagonist of the RNA polymerase activity.

29. The method of claim 19, wherein the compound that modulates the RNA polymerase activity is an agonist of the RNA polymerase activity.

30. A method of screening for compounds that modulate RNA polymerase activity in a continuous-read manner, comprising the steps of:
(a) contacting an RNA polymerase with an oligonucleotide template in a reaction mixture comprising an assay buffer and a fluorescent dye capable of binding double-stranded nucleic acid molecules, under conditions in which the RNA polymerase is active;
(b) adding a test compound to the reaction mixture;
(c) measuring the fluorescence of the reaction mixture; and
(d) determining whether the test compound modulates RNA polymerase activity.

31. The method of claim 30, wherein the RNA polymerase is a recombinant RNA polymerase.

32. The method of claim 30, wherein the RNA polymerase is the Hepatitis C virus (HCV) polymerase, NS5B.

33. The method of claim 31, wherein the NS5B polymerase is a recombinant NS5B polymerase.

34. The method of claim 33, wherein the recombinant NS5B polymerase comprises an amino acid sequence as set forth in SEQ ID NO: 2.

35. The method of claim 34, wherein the reaction mixture further comprises large unilamellar vesicles.

36. The method of claim 33, wherein the recombinant NS5B polymerase comprises an amino acid sequence as set forth in SEQ ID NO: 4.

37. The method of claim 30, wherein the fluorescent dye is an unsymmetrical cyanine fluorescent dye.

38. The method of claim 37, wherein the unsymmetrical cyanine fluorescent dye is excited at between 475 nm and 495 nm and dye fluorescence is detected at between 518 nm and 542 nm.

39. The method of claim 30, wherein the compound that modulates the RNA polymerase activity is an antagonist of the RNA polymerase activity.

40. The method of claim 30, wherein the compound that modulates the RNA polymerase activity is an agonist of the RNA polymerase activity.
